(19) United States
(12) Patent Application Publication
(10) Pub. No.: US 2013/0079768 A1
(43) Pub. Date: Mar. 28, 2013

(54) CATHETER WITH REVERSIBLE ADHESIVENESS, FOR STABILIZATION DURING TRANSCATHETER ABLATION BY MEANS OF RADIO FREQUENCY

(71) Applicants: Viviana De Luca, Bari (IT); Massimo Grimaldi, Bari (IT)

(72) Inventors: Viviana De Luca, Bari (IT); Massimo Grimaldi, Bari (IT)

(21) Appl. No.: 13/699,835

(22) PCT Filed: Jun. 7, 2011
(86) PCT No.: PCT/IB11/01248

§ 371 Date: Nov. 26, 2012

(30) Foreign Application Priority Data
Jun. 9, 2010 (IT) RM2010A000314

(51) Int. Cl. A61B 18/18

(52) U.S. Cl. CPC A61B 18/18
USPC 606/33

(57) ABSTRACT

A catheter that combines the delivery of radiofrequency, normally used in transcatheter ablation of cardiac arrhythmias, with a system providing temporary and reversible adhesion to heart tissue, so as to allow the stabilization of the catheter during breathing and pulsations of the heart, leading to more effective and more easily standardized radiofrequency lesions.

The present invention consists of a catheter that combines the delivery of radiofrequency, normally used in the transcatheter ablation of cardiac arrhythmias, with a system of temporary and reversible adherence to cardiac tissue which helps to stabilize the catheter itself during the acts of breathing and pulse of the heart, allowing the operator to make more effective and more easily standardized lesions.

### TECHNICAL FIELD

As is well known, the heart contracts continuously thanks to a system similar to an electrical system: some particular specialized structures conduct the stimulus that allows the heart to operate with a regular rhythm causing its contraction and allowing the pumping of blood in respect of the individual physiologies.

Sometimes, however, accelerated and irregular heart beats appear which are caused by faults in this electrical system; for example, there can be anomalous elements in the heart that can trigger abnormal re-entry electrical circuits.

In other cases, the electrical activity can be quite chaotic, giving rise to so-called atrial fibrillation.

### BACKGROUND ART

In the case of recurrent atrial fibrillation, it is customary, in recent years, to intervene using by the method of radiofrequency catheter ablation; this is a practice aimed at eliminating the hotbed of the cardiac arrhythmia or, where possible, interrupting the electrical circuit that causes it.

In practice, radiofrequency catheter ablation, consists of making a lesion, with a small burn in the heart, on that which is known as the “arrhythmogenic substrate” of the arrhythmia, that is the part of normal or diseased tissue in the heart which is the cause of the heart rhythm disorder.

This small burn (or thermal ablation) is carried out using a special catheter that is inserted in the heart cavity, usually through the femoral vein or artery.

Once in the heart cavity, having made the ablation catheter move on with the help of X-rays, the seat of the arrhythmia is sought thanks to electrical signals that the catheter itself records that are displayed on a monitor in front of the doctor.

Once the site of the arrhythmia has been identified, a small amount of electric power is applied through radiofrequency pulses, that heat the tissue near the tip of the catheter (or more precisely near the electrode(s) dispenser(s) wherever located on the surface of catheter) necrotizes the portion of tissue that is responsible for the arrhythmia, trying not to damage the surrounding healthy tissue.

Radiofrequency catheter ablation is now considered to be the first choice in the treatment of many supraventricular arrhythmias that often have as a common feature the fact that they recur and are not very responsive to medicine; the procedure is also used with patients who do not want to take drugs for long periods and who prefer to solve their problem radically. According to prior art, radiofrequency catheter ablation is presented as a sufficiently safe and effective method, especially when performed by experts; however, this is its main limitation, as its efficacy and the possible occurrence of complications, are largely related to the learning curve of the operator, or rather to his experience.

It can therefore be summarized by saying that this is a procedure which is highly “operator dependent”.

Because of this, it is currently difficult to standardize this process reaching an efficacy that is always repeatable and which is not dependant on the experience of the operator or his greater or lesser ability to perform this type of intervention.

In interventionist practice, the effectiveness of a series of transcatheter ablations carried out at a hospital is measured by the percentage of success, or lack of recurrence of the arrhythmia, and the percentage of complications that arise following the intervention.

These percentages are extremely variable and this shows just how variable the outcome of this type of intervention is.

The reasons for this difficulty in standardizing the thermal ablation procedure lie mainly in the fact that the effectiveness of the lesion made using the catheter depends on several factors, the most important of which are undoubtedly represented by the stability of contact between the catheter and heart tissue and, equally important, the pressure exerted by the catheter on the tissue during the distribution of radiofrequency.

Regarding the stability of contact between the catheter and cardiac tissue, it must be considered that the heart, because of its contractions, is a constantly moving structure and so once the portion of the tissue which must undergo ablation has been identified, the catheter should be placed on the affected part and kept firmly in place even during cardiac contractions.

In clinical practice, the catheter tends, however, to move according to the continuing contractions of the heart, which sometimes hinders the execution of the lesion at a given point.

Here, therefore, only the skill of the operator can effectively overcome this problem, resulting in a precise and effective lesion.

The other problem that usually results in the lowering of the effectiveness of a transcatheter ablation, is represented by a lack of pressure of the catheter on the surface of the tissue to be subjected to thermal ablation.

Generally, when a lesion is made with a catheter which distributes radiofrequency, the lesion is made all the more effective the greater the pressure exerted locally by the electrode distributor of the catheter on the portion of tissue to be removed.

Because of this, if a catheter is not well stabilised on the point at which to carry out the lesion, the pressure on this point will probably not be suitable and so there is the risk of making an ineffective lesion as modest pressure prevents the lesion from reaching the necessary depth. To try to overcome the above inefficiency, that is to cause a lesion which is sufficiently deep, the operator often has to increase the power (watts delivered by the electrode) which may cause a sudden increase in the temperature of the tissue, resulting in the formation of water vapour, which in turn can cause micro-explosions and extremely dangerous cavitations and often with disastrous consequences.

The main purpose of the present invention is to provide a catheter which allows the standardization of transcatheter radiofrequency ablation, making the results of this type of intervention more replicable.

Another important aim is to provide a catheter whose use would result in a more effective intervention and, above all, that this result can be made less dependant on the experience of the operator or his greater or lesser ability to perform this type of intervention.

For these purposes the invention solves the problem of the stabilization of the catheter on the portion of tissue to be subjected to thermal ablation, even during continuous contractions of the heart muscle which tend to move the catheter.

The consequence of this is that the catheter of the present invention allows the continuous delivery of radiofrequency to the same point and also allows the operator to exert adequate pressure on the portion of cardiac tissue to undergo ablation during the entire process of the delivery of radiofrequency, thus making lesions made with this technique more effective and more easily standardized.

### BRIEF DESCRIPTION OF DRAWINGS

FIG. 1) side view of the catheter with the element extended, in execution with four electrodes positioned on the lateral surface;

FIG. 2) the catheter in curved position within the cardiac cavity;

FIG. 3) adherence of the catheter to the area of cardiac tissue on which the unipolar radiofrequency lesion is to be carried out;

FIG. 4) cross section of the catheter;

FIG. 5) execution of the catheter in which the pole of stabilization on its tip, is made integral to it by means of a connection;

FIG. 6) execution of the catheter with four electrodes one pair of which distributes bipolar radiofrequency.

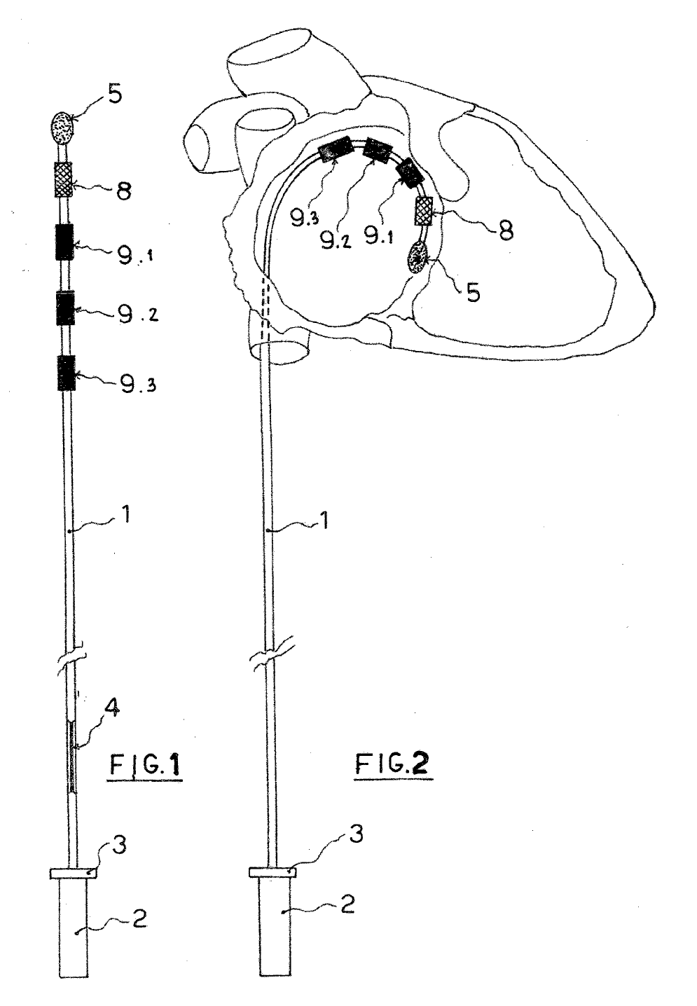

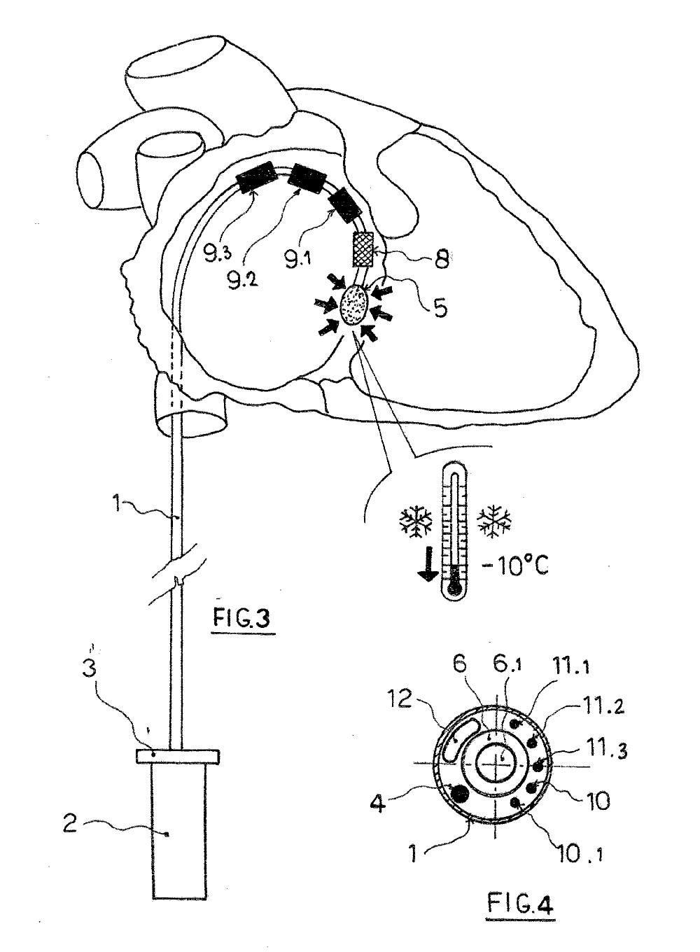

### DISCLOSURE OF INVENTION

The catheter in this invention is substantially composed of a flexible hollow element 1, which, like catheters of known art, has a handle 2 at the lower end with a plunger 3 through which one acts on a pull-wire 4 which is coaxial to and inside the hollow element 1, whose ends are respectively connected to the tip of the catheter and the plunger 3 so that by acting on the latter moving it away from the operator, the tip of the catheter is called back and folds the flexible hollow element 1 by the desired amount; on the contrary, pulling the plunger 3 towards the operator the flexible hollow element is extended.

The main innovation introduced by this invention is to provide the distal end of the catheter, or its tip, with a pole of stabilization 5, through which the catheter is temporarily and reversibly adhered to the heart tissue.

In this way, when the point on which to practice the lesion delivering radiofrequency has been identified, the catheter is placed on the tissue in the most suitable way and is stabilized by adhering the pole 5 to the tissue.

The adherence of the pole of stabilization 5 to a generic point in the cardiac tissue, allows the maintenance of the position of the catheter during cardiac contractions, which would tend instead to move it, especially when its positioning is not sufficiently stable.

The fact that the catheter is stabilized by the adhesion of the pole 5 to a generic point of the heart tissue, allows the operator to apply the necessary pressure on the catheter in order to achieve a sufficiently effective lesion.

The immediate consequence of this, is that the operator can distribute the proper radiofrequency power without having to resort to dangerous increases, which were often necessary to achieve a deeper lesion that compensated for the lack of stability or sufficient contact pressure between the catheter and tissue to be removed.

It is anticipated that the adhesion of the pole of stabilization 5 must be temporary and reversible, i.e. it must allow the operator to remove the catheter from the portion of tissue on which he carried out the lesion, to then reposition and stabilize it in another place.

The temporary adhesion of the pole of stabilization 5 to a generic portion of heart tissue, is created by lowering the temperature until it reaches a value of several units below 0° centigrade.

Bringing the pole 5 in contact with a generic portion of the tissue and then lowering its temperature to a value between −10 ° and −30 ° centigrade, a strong bond is created between the pole 5 and the tissue with which it is in contact, which allows the stabilization of the catheter in any position, making it sufficiently insensitive to the continuous contractions of the heart muscle that tend to move it

It is not advisable to go beyond the above-mentioned temperature range, because an excessive cooling of the pole of stabilization 5 would produce a sort of cryoablation, resulting in irreversible damage to the tissue at the point of contact with the pole 5.

Only by keeping the pole of stabilization 5 at a temperature between −10 ° and −30 ° centigrade, is an entirely reversible adhesion achieved causing no damage to the portion of tissue on which the adhesion is carried out.

In practice, once the site of the arrhythmia has been identified, the operator positions the catheter in the most suitable way bringing the pole 5 in contact with the heart tissue and cooling it in order to create the adhesion with the tissue and stabilizes the catheter; at this point the operator can apply the necessary pressure on the tissue and deliver the radiofrequency through the electrode dispenser(s) wherever they are positioned on the surface of the catheter, heat and necrotize the portion of tissue that is responsible for the arrhythmia. To be able to temporarily cool down the pole of stabilization 5, the hollow element 1 is passed through by a tube 6 for the transit of a suitable compound that can quickly lower the temperature of the pole 5, for example nitrous oxide.

The lower end of the tube 6 is indirectly connected to appropriate equipment capable of delivering on command a variable amount of nitrous oxide; the opposite end of the tube 6 terminates, instead, at the pole of stabilization 5, where the nitrous oxide expands, bringing the temperature of the pole 5 to the desired value.

The greater or lesser amount of nitrous oxide sent and conveyed through the tube 6, determines the degree of cooling of the pole of stabilization 5.

When the operator wants to achieve adhesion between the pole 5 and the heart tissue, he simply has to send, using a dedicated remote control, a certain amount of nitrous oxide to the pole 5; the latter, instantly cooling, creates the adhesion with the tissue with which it is in contact for as long as the nitrous is sent, then it ceases when it is no longer fuelled by nitrous oxide.

In this way an adhesion is created between the pole of stabilization 5 and the tissue with which it is in contact, which is reversible an infinite number of times.

In the proposed execution, said tube 6 also provides for the recovery of nitrous oxide after it is sent to the pole 5; the tube 6 has a tube for this purpose which is internal and coaxial 6.1, so nitrous oxide can be sent through a portion of the annular tube between the inner surface of tube 6 and outer surface of the coaxial tube 6.1, then to be aspirated only through the coaxial tube 6.1 after the nitrous oxide has been used.

As previously mentioned, the catheter of the present invention is able to perform radiofrequency ablation, by means of a small amount of electric power that heats the electrode(s) wherever they are positioned on the surface of the catheter, burning the portion of tissue that is responsible for the arrhythmia.

This implies that the catheter has at least one electrode that delivers unipolar radiofrequency;

in the execution in FIG. 1), an electrode 8 can be observed which is intended to provide unipolar radiofrequency, that is punctiform.

In the proposed execution, the electrode 8 is arranged in a ring outside the lateral surface of flexible hollow element 1 of the catheter.

On the lateral surface of the flexible element 1, there are three electrodes, 9.1, 9.2 and 9.3, each forming an electric dipole with the electrode 8 for so-called sensing, that is for the acquisition of information related to the arrhythmias; the electronic information recorded by the electrode 8 is transmitted by a thin wire 10.1 housed within the flexible hollow element 1. In order to supply power to the electrode 8, to enable it to deliver radiofrequency, the flexible hollow body is also passed through by an electric wire 10.

[symbol] to the heart respectively through the wires 11⁹.1, 11.2 and 11.3 that also pass through the longitudinal cavity of the flexible element 1.

The electrode 8 is then irrigated with a suitable liquid, in order to allow the effective cooling of the electrode itself and the tissue with which it is in contact during the entire process of the delivery of radiofrequency; for this purpose the hollow body is passed through by a tube 12 also ending at the electrode 8 and which is for the transit of the irrigation liquid that then comes out of the appropriate holes on the surface of the electrode 8.

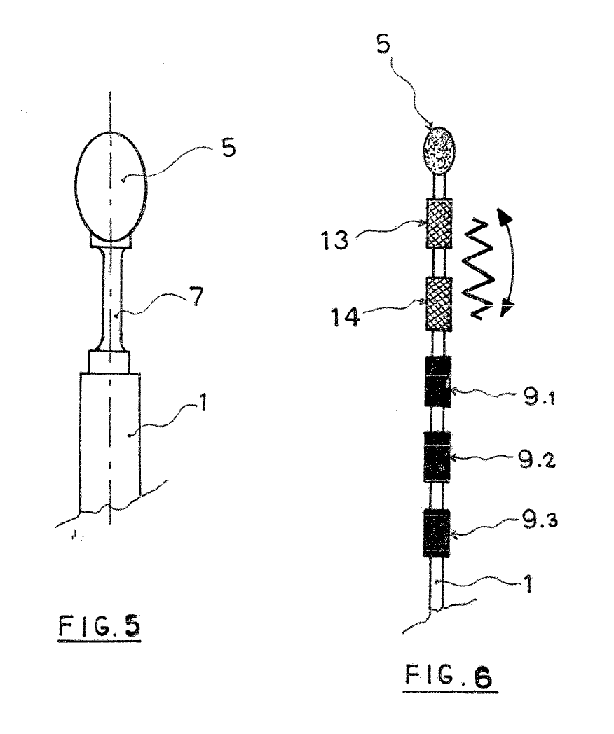

In an improved execution (FIG. 5), the pole of stabilization 5 is made integral to the distal end of the flexible element 1, that is its tip, by means of a flexible coupling 7.

This flexible coupling 7 enables the smallest reciprocal movements between the pole of stabilization 5 and a flexible element 1.

This allows the operator to position the flexible element 1 in the most appropriate way, to prevent that movements of the flexible element 1 after the adhesion between the pole 5 and the tissue cause dangerous tractions on the latter that could, at the outside, also cause lacerations at the point of adhesion of the pole 5.

The flexible coupling 7 is therefore made from a suitable semi-rigid material capable of ensuring at the same time, rigidity, to allow the stabilization of the entire catheter, and flexibility to allow small reciprocal movements between the pole 5 and the flexible element. The flexible coupling 7 is also hollow to allow the passage of the tube 6 (and its coaxial tube 6.1) that feeds the nitrous oxide (or other suitable substance) to the pole of stabilization 5. In the execution in FIG. 6), the catheter has four electrodes of which the pair 13 and 14, in accordance with what is claimed in the international patent application PCT/IT2008/000397, delivers bipolar radiofrequency, that is the radiofrequency which is delivered from the distal electrode 13 (which acts as a negative pole) to the proximal electrode 14 (positive pole), so as to create a linear rather than punctiform lesion.

The possible presence of a greater number of adjacent electrodes which deliver bipolar radiofrequency (e.g. four or six), would allow linear lesions of greater length to be carried out. This representation is illustrated only to show an execution that integrates a solution proposed in a patent belonging to the known art.

Ultimately, the union of a radiofrequency distributing electrode 8 and a pole 5 distributing cold, allows for a catheter that uses the cold to create a temporary and reversible adhesion, stabilizing the catheter on the tissue which is to undergo ablation, and radiofrequency to achieve a more efficient lesion and greater speed in carrying out that lesion.

In this way the intervention of radiofrequency catheter ablation can be standardized, achieving greater effectiveness which is not dependent on the subjectivity of the intervening operator.

1. Catheter with reversible adhesiveness, for stabilization during transcatheter ablation by means of radio frequency comprising a flexible hollow element with a grip at the lower end with a plunger through which it acts on a pull-wire which is coaxial to and inside the hollow element, an electrode suitably and conveniently placed on the outside surface of the flexible hollow element capable of delivering unipolar radiofrequency and for this powered by an electric wire inside the flexible hollow element, one or more electrodes for sensing, for example powered respectively by electric wires inside the lengthwise cavity of the flexible hollow element, each electrode for sensing forming an electric dipole with the electrode whose recorded information is transmitted by an electric wire housed within the flexible hollow element, the electrode finally being irrigated during the delivery of radiofrequency with a suitable liquid carried by a tube which is also housed in the longitudinal cavity of the flexible hollow element and ends at the electrode from which the liquid flows through appropriate holes on its surface, said catheter further comprising a pole of stabilization which can adhere temporarily and reversibly to a generic tissue with which it is preliminary in contact with, said adherence taking place with the cooling of the pole of stabilization until it reaches a temperature between 0° and −30° centigrade, obtained by passing a small tube ending in the pole through the flexible hollow element which causes the release, at the command of the operator, of a suitable quantity of a compound that can quickly lower the temperature of the pole, which in turn comes from a device to which the lower end of the tube is directly or indirectly connected, a tube inside the longitudinal cavity of the flexible hollow element, ensuring on command the recovery by aspiration of the compound used for cooling the pole of stabilization, the latter being wherever placed on the catheter.

2. Catheter with reversible adhesiveness according to claim 1, wherein in a variation of execution the pole of stabilization is made integral to the distal end of the flexible hollow element, or to its tip, through a flexible coupling which can make mutual movements possible between said pole of stabilization and the flexible hollow element, the flexibility of the connection being obtained through the use of a suitable material or, alternatively or in combination, by means of a special realization realisation of the same, in both cases it being ultimately possible to realize a lengthwise cavity in said flexible connection for the passage of the small tubes.

3. Catheter with reversible adhesiveness according to claim 1, further having two or more poles of stabilization wherever they are positioned on the external surface of the catheter, each of them being supplied by a tube carrying a suitable compound for cooling, the catheter also having one or more small suction tubes for the recovery on command of the compound used to cool the two or more poles of stabilization, the supply and recovery tubes being housed inside the longitudinal cavity of the flexible hollow element.

4. Catheter with reversible adhesiveness according to claim 1, further comprising being equipped with two or more electrodes which distribute radiofrequency whose electrical power is supplied by an equivalent number of electrical wires housed inside the lengthwise cavity of the flexible hollow element.

5. Catheter with reversible adhesiveness according to claim 1, wherein the pole(s) of stabilization and electrode(s) which distribute(s) radiofrequency can be placed anywhere and anyhow on the outer surface of the catheter, or from the top to the generic point of the external lateral surface of the flexible hollow element.

6. Catheter with reversible adhesiveness according to claim 1, wherein the compound that can quickly lower the temperature of the pole is nitrous oxide.
